(19) United States
(12) Patent Application Publication
(10) Pub. No.: US 2011/0238042 A1
(43) Pub. Date: Sep. 29, 2011

(54) Drainage Catheter with One-Way Valve

(75) Inventors: Michele G. Davis, Forsyth, GA (US); Jaime Ritter, Snellville, GA (US)

(73) Assignee: C. R. BARD, INC., Murray Hill, NJ (US)

(21) Appl. No.: 12/523,639

(22) PCT Filed: Oct. 2, 2008
(86) PCT No.: PCT/US2008/078553

§ 371 Date: Feb. 18, 2011

Related U.S. Application Data
(60) Provisional application No. 60/976,930, filed on Oct. 2, 2007.

(51) Int. Cl. A61M 27/00

(52) U.S. Cl. 604/544

(57) ABSTRACT

Systems and methods that serve to drain a bodily fluid are provided The medical device includes a lumen connecting between the proximal end and the distal end The lumen is preferably adapted to drain fluid from the distal end to the proximal end The device may also include a pressure sensitive valve adapted for insertion and placement in the lumen The pressure-sensitive valve may be pressure sensitive wherein, when pressure at the inflow port rises to a predetermined threshold, the pressure-sensitive valve opens The lumen may also be configured whereby, when pressure at the inflow port drops to a second predetermined threshold, the pressure-sensitive valve closes In some embodiments, the valve may be unidirectional and not sensitive to any substantial pressure threshold but may open at any substantially non-zero pressure applied in the appropriate direction.

The present application claims priority to U.S. Provisional Application No. 60/976,930, filed Oct. 2, 2007, the disclosure of which is incorporated herein by reference in its entirety.

This patent application relates to drainage catheters. More specifically, this patent application relates to preventing infection when using urinary tract catheters, but may apply to preventing infection when using any suitable drainage catheter that serves to drain bodily fluids.

The most commonly known urinary tract catheter is the Foley catheter. The Foley catheter is a catheter device usually made out of elastomeric material, which is for urine drainage and which is installed with its distal end in the bladder of the patient. When the distal end of the catheter has been advanced into the bladder, sterile water may be caused to flow along a lumen, from the proximal to the distal end of the catheter, and into a balloon at the distal end of the catheter This balloon retains the distal end of the catheter in the bladder and allows a second lumen (for drainage) in the catheter shaft, open to the bladder at the distal end of the shaft, to drain urine from the bladder to the proximal end of the catheter.

There are three primary routes of infection for catheter-associated urinary tract infection. The first route of infection is upon insertion of the catheter in a patient. Contamination of the distal tip of the catheter may cause this infection.

A second route of infection may be from retrograde migration of microorganisms from the drainage bag. Microorganisms may migrate into the bladder of the patient or into some other internal area of the patient via the drainage lumen described above. This route of infection may be referred to hereinafter as “intraluminal” contamination.

A third route of infection may be via migration of bacteria on the outside of the catheter. Bacteria may migrate on the outside of the catheter into the urethra of the patient, the bladder of the patient or into some other internal area of the patient. This route of infection may be referred to hereinafter as “extraluminal” contamination.

It would be desirable to provide systems and methods that reduce the risk of catheter-associated urinary tract infection.

Another embodiment of the invention relates to controlling urine drainage from the bladder.

When a patient is catheterized for a long period of time, the bladder may lose muscle tone. When use of the Foley catheter is discontinued, the patient may frequently suffer from incontinence.

Conventional practice is to “re-train” the bladder during the catheterization period by clamping the catheter using a clamp external to the body, letting urine accumulate until the patient verbalizes, or otherwise indicates, discomfort or urge, and, thereafter, releasing the clamp to let the bladder drain.

However, this practice may damage the catheter and can also lead to adverse events such as infection and pain.

Thus, it would also be desirable to implement a Foley catheter that includes a system of training the bladder or, at the least, promotes muscle tone in the bladder during the catheterization period.

It is an object of the disclosure to provide systems and methods that reduce the risk of catheter-associated urinary tract infection.

In one embodiment of the disclosure that suitably obtains reduced intraluminal contamination, a one-way valve may be placed inside the drainage lumen in the catheter or at some other suitable location in the drainage tubing that is coupled to the catheter. This valve may prevent migration of microorganisms through the lumen into the bladder of the patient or into some other internal area of the patient. The one-way valve, alternatively referred to herein as a unidirectional valve, may allow for passing fluid in one direction and obstructs fluid passing in an opposite direction.

A one-way valve according to the disclosure may be implemented according to numerous different designs. Suitably, a one-way valve according to the disclosure may allow the passage of urine “downstream” into the drainage bag but prevents the passage of microorganisms “upstream” past the valve. The presence of the valve may act as a barrier regardless of the material of the valve. The valve may have a relatively slick surface to prevent, or at least reduce, bacterial attachment thereto. Alternatively, or additionally, the valve may also be coated with an antimicrobial agent to kill bacteria and/or prevent bacterial proliferation.

A further advantage of such a valve may be that, because the valve is non-patient contacting, certain relatively more potent antimicrobials including but not limited to organic acid metal salts, such as salts comprising phosphoric acid esters and zinc metal, particularly materials that can provide a stabilizing effect to polymers, and, in particular, a heat stabilizing effect to PVC and other polymer materials for use in a urine collection system, or patient-contacting antimicrobials, could be used to coat the valve. Alternatively, these or other antimicrobials could be molded into the valve. Such antimicrobials, which may be used to coat the valve or be molded into the actual material used to form the valve, are described in greater detail in commonly-assigned U.S. Pat. No. 6,716,200 which is hereby incorporated by reference herein in its entirety.

In one embodiment of the invention, the one-way valve may be placed in the funnel of the catheter. The funnel is adapted to couple drainage tubing to the catheter. The drainage tubing may serve to couple the catheter to a urine receptacle. The lumen in the catheter typically narrows from the relatively wider end of the funnel towards the narrow portion of the funnel. The narrow portion of the funnel is closer to the tip of the catheter. One benefit of placing the valve in the funnel of the catheter is that the funnel helps to hold the one-way valve in place. Nevertheless, at least because the function of the valve is to prevent interluminal contamination, the valve can be placed almost anywhere along the drainage circuit—e.g., at any point in the catheter, in the drainage tubing, etc.

An added benefit of a one-way valve according to the invention may be that the valve may prevent urine from passing from the bag to the patient's bladder if the bag is mishandled.

A further concept of the invention involves placing a pressure-sensitive valve at some point along the drainage system. Placement of the valve may be within the catheter drainage lumen, the lumen of the drainage tubing, within the junction of the catheter and drainage tubing or even the junction of the drainage tubing to the drainage bag. The pressure sensitive valve can remain closed until a certain level of pressure is obtained. Once a threshold level of pressure on the valve is met, the valve opens and allows urine to drain. This allows the bladder to fill and then empty, thereby maintaining tone of the bladder, without the repeated, and often painful and unhealthy, use of an external clamp.

According to various embodiments, a pressure-sensitive valve according to the disclosure may be placed near the distal tip of the catheter—i.e., the portion of the catheter that resides in the patient's bladder. This placement—i.e., near to the distal tip of the catheter—allows the bladder to retain the majority of the accumulated urine. This placement may also obtain a further benefit of preventing the retention of urine in the drainage tube.

### BRIEF DESCRIPTION OF THE DRAWINGS

For the purposes of illustrating the various aspects of the invention, there are shown in the drawings forms that are exemplary, it being understood that the invention is not limited to the precise arrangements and instrumentalities shown.

FIG. 1 is a longitudinal diametrical section through a conventional Foley catheter.

FIG. 2 is a longitudinal diametrical section through a Foley catheter according to the invention.

FIG. 3 is a longitudinal diametrical section through the proximal end of the FIG. 2 catheter showing the one-way valve implemented in the funnel of the Foley catheter.

FIG. 4 is a schematic view of a conventional urine collection system

FIG. 5 is a schematic diagram of a catheter drainage tube implementing a pressure sensitive valve according to the invention.

FIG. 6 is an illustration of a pressure sensitive valve for use in systems and methods according to the invention.

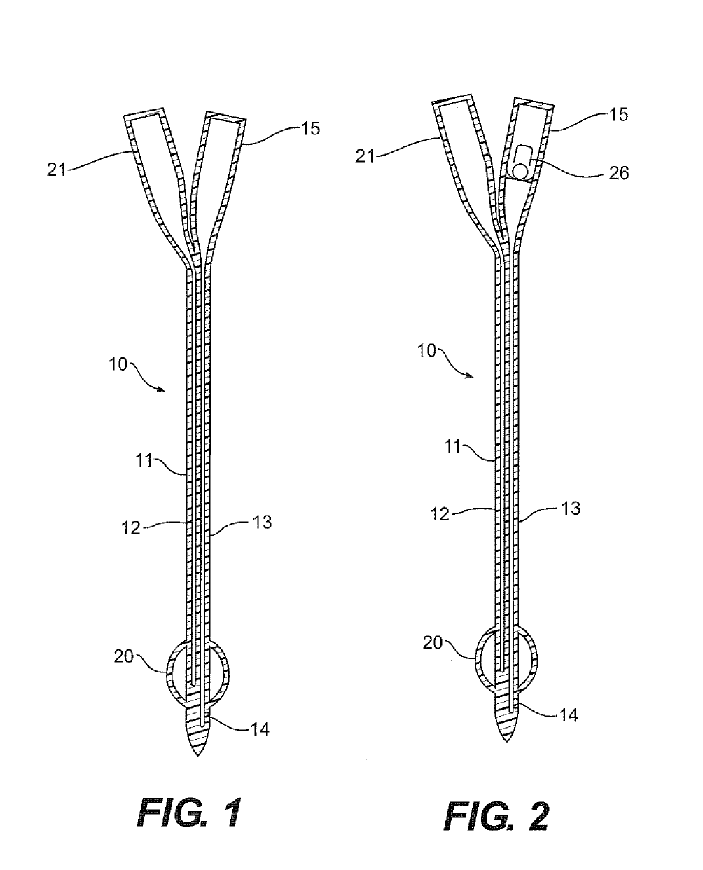

FIG. 1 shows a known Foley catheter. The catheter 10 includes a shaft 11 of latex rubber, silicone, or any other suitable material, which defines a balloon inflation lumen 12 and a drainage lumen 13. Drainage lumen 13 extends from a distal drainage port 14 to a drainage bag coupling element 15, referred to, in the alternative, herein for purposes of this application as a funnel. The inflation lumen 12 connects a chamber 20 at the distal end of the catheter, but proximal of the drainage port 14, with intake port 21 at the proximal end of the device.

It should be understood that, for the purposes of this application, non-essential details of the Foley catheter described above are for background purposes only and should not be interpreted to limit the disclosure.

FIG. 2 shows a longitudinal diametral section through a Foley catheter according to the disclosure. A one-way valve according to the invention is shown schematically at 26 (an exemplary one-way valve is shown in more detail in FIG. 6).

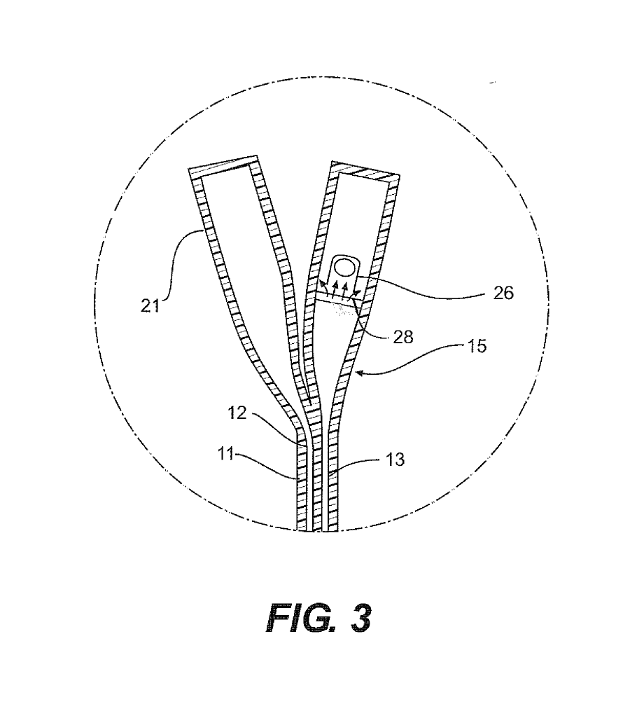

FIG. 3 shows a longitudinal diametral section through the proximal end of the catheter of FIG. 2. Retention fingers, which may be barbed, hooked, bent, or otherwise deformed, are shown at 28. Retention fingers serve to affix at least a portion of valve 26 to funnel 15 such as to form a substantially leak-proof seal about the circumference of at least a portion of valve 26. Alternatively, one-way valve 26 may be friction fit, or otherwise affixed, within funnel 15 (or at some other suitable location along the drainage circuit).

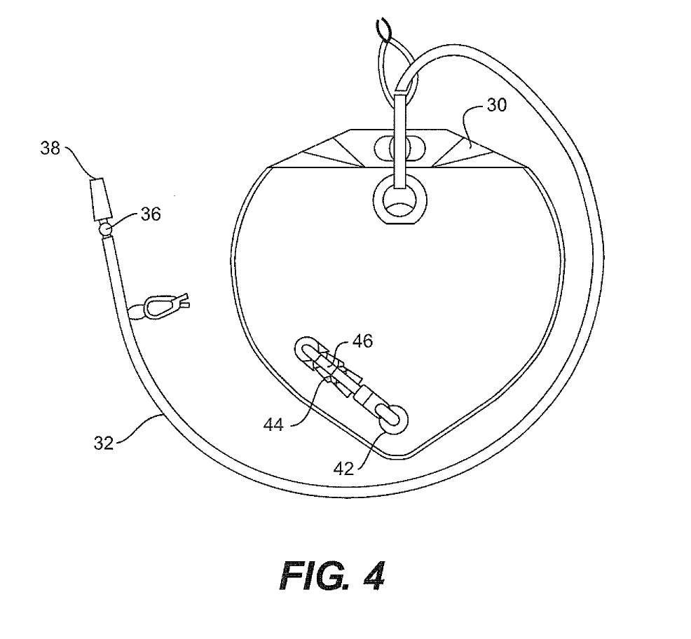

FIG. 4 shows a typical urine collection system for use with the catheters shown in FIGS. 1-3. The system may include a urine collection bag, generally indicated as 30, a drainage tube, generally indicated as 32, which is connected to a sampling port 36 and a catheter connector 38 for connecting tube 32 to a funnel of the catheters shown in FIGS. 1-3, or other suitable catheter. A typical collection bag 30 also includes an outlet tube port 42 which has an outlet tube/connector mechanism 44 for emptying collection bag 30 through an outlet tube 46.

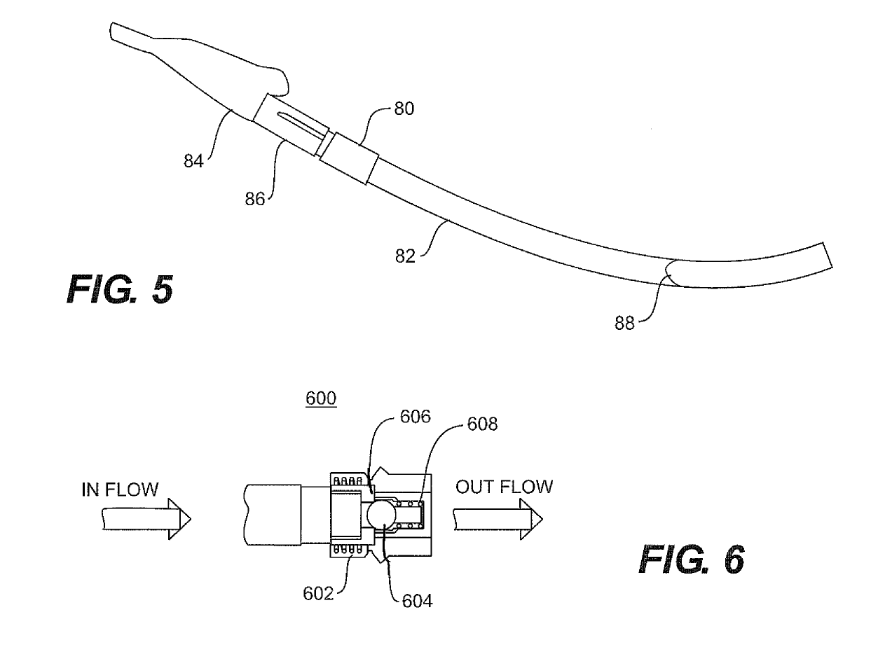

FIG. 5 shows a catheter drainage tube 82 implementing a valve 80 according to the invention. FIG. 5 also shows funnel 84, catheter coupler 86 and waterline 88.

Valve 80 may be unidirectional and/or pressure-sensitive. As described above, valve 80 may remain closed inside the catheter until a certain level of pressure is obtained. Once a pre-determined threshold level of pressure on the valve is met, the valve opens and allows urine to drain. Thereafter, valve 80 closes and allows the bladder to fill again. This operation allows the bladder to fill and then empty in a cadence and rhythm that is similar to the operation of a fully operational, non-catheretized, bladder. Such operation maintains tone of the bladder without the repeated use of an external clamp to manually institute a rhythm on the operation of the bladder. It can be seen from waterline 88 that valve 80 prevents the retrograde flow of fluid.

Valve 80 according to the disclosure may be placed near the distal tip of the catheter in order to allow the bladder to retain the majority of the urine prior to the opening of pressure sensitive valve 80. This placement also prevents retention of urine in the drainage tube. A further advantage of such a placement of valve 80 near the distal tip of the catheter is that such a placement prevents retrograde flow of urine from the catheter into the bladder.

In one embodiment of the invention, valve 80 maybe allow urine to pass in a hysteretic fashion such that, when the urine pressure obtains a predetermined threshold, the valve cracks open and does not close again until the bladder is substantially empty. Thus, the pressure inside the bladder may rise to a peak—i.e., the predetermined urine pressure threshold—and then drop sharply off, at a slope that is greater than the slope by which the pressure built, until a sufficient amount of urine is emptied from the bladder, and the urine pressure has dropped to a second pre-determined threshold. The second pre-determined threshold may be substantially lower than the first pre-determined urine pressure threshold. Thereafter, at the second predetermined threshold, valve 80 may close, and the bladder may begin to fill again.

In this embodiment of the disclosure, a hysteretic pattern may occur wherein the valve opens at the pre-determined pressure threshold, as the bladder substantially empties the urine pressure chops far below the threshold, and then the valve closes and the bladder pressure begins to build pressure again. Other embodiments of the disclosure may implement multiple valves, or, alternatively, a more-complex valving scheme.

As described above, the attachment of any of the valves according to the invention may be obtained by fiction fit of the valve into a suitably shaped portion of the drainage circuit of the apparatus according to the invention. In alternative embodiments of the invention, the attachment of the valve to any suitable portion of the drainage apparatus may be obtained by using hooked fingers—i.e., the ends of the fingers are bent—or barbed fingers—i.e., the ends of the fingers have small projections that secure the fingers to their respective surface of engagement—or a combination of the two, spaced substantially circumferentially around the outer annulus of the unidirectional valve.

In certain embodiments, the fingers may be formed from an elastic material. In these embodiments, the fingers may be stretched or otherwise manipulated to engage the drainage tubing, catheter or any other portion of the drainage circuit and retain the portion attached thereto with the hooked or barbed ends of the fingers. The elasticity of the fingers then causes the fingers to snap back such that the tissue graft is secured to the drainage tubing, catheter, etc.

Alternatively, the fingers may be formed from relatively non-elastic material such as stainless steel, etc. In these embodiments, the fingers are deformed to properly engage the appropriate section of the drainage circuit and then mechanically crimped to seal the connector to the surrounding tubing. In any case, the barbs or fingers preferably engage the surrounding tubing and provide a substantially leak-proof seal about the relevant annulus of the surrounding tubing.

FIG. 6 shows an illustration of one possible valve 600 that may be used in systems and methods according to the disclosure. Valve 600, commonly known as a check valve, includes body 602, ball 604, seat 606 and spring 608. Check valves are unidirectional flow control devices used to eliminate potential damage caused by backflow. Thus, flow in the desired direction causes a mechanism, such as ball 604, to open the valve, while backflow causes the valve to close. Spring-loaded check valves, such as valve 600, include a spring 608 to open and close the valve. In all check valves, the “cracking pressure” is the minimum p.s.i. (pounds per square inch) required to open a valve. Suitable valves for use with systems according to the disclosure may include valves produced by the Lee Company of Westbrook, Conn., but many other valves are available that may suit the specifications of the embodiments of the invention.

When valve 600 includes spring 608, the cracking pressure is relatively higher than when valve 600 does not include spring 608. This is because spring 608 exerts a force on ball 604 and maintains ball 604 in seat 606 until the force from the spring is met and overcome by the fluid flow. Otherwise, ball 604 acts to allow fluid flow in one direction and prevent fluid flow in the other direction.

As such, when a spring is not included in valve 600 (as shown in FIGS. 2 and 3), valve 600 may be used for the one-way valve embodiment of the disclosure which prevents backflow from occurring in the catheter. When a spring, such as spring 608, is included in valve 600 then the pressure-sensitive valve embodiment of the disclosure, which may be unidirectional as well, is described. It should be noted that in certain embodiments of the pressure-sensitive valve embodiment of the invention, the cracking pressure may be higher than the pressure required to close the valve, once opened. As such, the valve may open at a first, higher, pressure, remain open while urine drains, and then close at relatively lower pressure.

Thus, systems and methods for reducing the risk of catheter-associated urinary tract infection are provided. Furthermore, systems and methods of maintaining bladder tone are provided.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A medical device that drains bodily fluid, said device comprising:
a proximal end and a distal end;
a lumen connecting between the proximal end and the distal end, the lumen adapted to drain fluid from the distal end to the proximal end, the lumen having a fluid inflow port at the lumen's distal end and a fluid drain coupling at the lumen's proximal end; and
a unidirectional valve adapted for insertion and placement in the lumen, said unidirectional valve that allows for fluid passing from the distal end to the proximal end and obstructs fluid from passing from the proximal end to the distal end.

2. The medical device of claim 1, the fluid drain coupling that houses the unidirectional valve.

3. The medical device of claim 1, further comprising:
a receptacle to collect urine;
an inlet connected to the receptacle, the inlet configured to permit the passage of urine therethrough; and
drainage tubing comprising a proximal end and a distal end, the proximal end connecting to the inlet, the distal end comprising a catheter connector, the catheter connector for connecting the drainage tubing to the fluid drain coupling.

4. The medical device of claim 1, wherein, when urine pressure builds to a pre-determined threshold, the unidirectional valve opens and allows mine to pass therethrough.

5. The medical device of claim 4, wherein the unidirectional valve closes when the mine pressure drops to a second pre-determined threshold.

6. The medical device of claim 1, wherein the unidirectional valve is coated at least in part with an antimicrobial agent.

7. The medical device of claim 1, wherein the unidirectional valve is formed at least in part with an antimicrobial agent.

8. A medical device for collection of urine, said device comprising:
a receptacle to collect urine;
an inlet connected to the receptacle, configured to permit the passage of mine therethrough; and
drainage tubing comprising a proximal end and a distal end, the proximal end connecting to the inlet, the distal end comprising a catheter connector, the catheter connector for connecting the drainage tubing to a fluid drain coupling of a Foley catheter, the drainage tubing further comprising a unidirectional valve, said unidirectional valve that allows fluid to pass from the distal end to the proximal end and obstructs fluid from passing from the proximal end to the distal end.

9. The medical device of claim 8, the catheter connector that comprises the unidirectional valve.

10. The medical device of claim 8, the catheter connector that houses the unidirectional valve.

11. The medical device of claim 8, wherein the unidirectional valve is located on the distal end of the drainage tubing.

12. The medical device of claim 8, further comprising a Foley catheter, the catheter comprising:
a proximal end and a distal end; and
a lumen connecting between the proximal end of the catheter and the distal end of the catheter, the lumen adapted to drain fluid from the distal end of the catheter to the proximal end of the catheter, the lumen having a fluid inflow port at the lumen's distal end and a fluid drain coupling at the lumen's proximal end, and wherein the fluid drain coupling is coupled to the catheter connector.

13. The medical device of claim 8, wherein the unidirectional valve is coated at least in part with an antimicrobial agent.

14. The medical device of claim 8, wherein the unidirectional valve is formed at least in part with an antimicrobial agent.

15. A medical device that drains bodily fluid, said device comprising:
a proximal end and a distal end;
a lumen connecting between the proximal end and the distal end, the lumen adapted to drain fluid from the distal end to the proximal end, the lumen having a fluid inflow port and a fluid drain coupling; and
a pressure-sensitive valve adapted for insertion and placement in the lumen, said pressure-sensitive valve characterized such that, when pressure at the inflow port rises to a predetermined threshold, the pressure-sensitive valve opens, and when pressure at the inflow port drops to a second predetermined threshold, the pressure-sensitive valve closes.

16. (canceled)
